(19) United States
(12) Patent Application Publication
(10) Pub. No.: US 2021/0276905 A1
(43) Pub. Date: Sep. 9, 2021

(54) MICROBIAL TREATMENT FOR LIVESTOCK WATER SYSTEMS

(71) Applicant: New Life Biosciences LLC, The Woodlands, TX (US)

(72) Inventor: Marc Rodriguez, The Woodlands, TX (US)

(21) Appl. No.: 16/849,823

(22) Filed: Apr. 15, 2020

Related U.S. Application Data
(60) Provisional application No. 62/986,499, filed on Mar. 6, 2020.

(51) Int. Cl. C02F 3/34; C02F 3/10

(52) U.S. Cl. CPC C02F 3/348; C02F 2101/306; C02F 3/10

(57) ABSTRACT

A method and composition for treating drinking water for animals is disclosed. A method for treating drinking water for animals includes providing a plurality of sporulated microbes to metabolize an organic matter in the drinking water. A composition for treating animal drinking water includes a plurality of sporulated microbes.

The present application claims priority as anon-provisional to U.S. Provisional Application 62/986,499, filed on Mar. 6, 2020, presently pending. The contents of the application are hereby incorporated by reference.

### FIELD

The present disclosure relates to systems and methods for treating drinking water. More specifically, the disclosure relates to systems and methods for treating drinking water to be consumed by animals.

### BACKGROUND

Livestock such as cattle, horses, pigs, sheep, chickens, etc., require regular access to drinking water. Systems used to provide drinking water to livestock may be circulating water systems, a water supply to a tank, or a water tank, and can include a holding tank, trough or ball waterer for the livestock to access the drinking water.

The drinking water of livestock watering systems is subject to contamination. Contaminants may include algae, bacteria, protozoan parasites, helminth parasites, zoonotic pathogens and/or coliform bacteria. It is important to clean the drinking water to maintain livestock health. Typically cleaning animal drinking water is accomplished by periodic cleaning of watering systems, but this can be labor and time-intensive. For example, a livestock watering tank may need to be regularly drained and scrubbed to maintain a suitable and clean drinking water supply.

An object of the present disclosure is to provide a method and composition for cleaning animal drinking water that is effective and simple to carry out.

### SUMMARY

A further understanding of the functional and advantageous aspects of the invention can be realized by reference to the following detailed description and drawings.

An object of the present disclosure is to provide a method and composition for treating drinking water for animals.

Thus, by one broad aspect of the present invention, a method for treating drinking water for animals is provided, including providing a plurality of sporulated microbes to metabolize an organic matter in the drinking water.

By a further broad aspect of the present invention, a composition for treating animal drinking water is provided, including a plurality of sporulated microbes.

### BRIEF DESCRIPTION OF THE DRAWINGS

Embodiments disclosed herein will be more fully understood from the following detailed description taken in connection with the accompanying drawings, which form a part of this application, and in which:

FIG. 1 is a flow chart of an embodiment of the system showing an overview of a method of treatment of livestock water, according to one embodiment of the invention.

FIGS. 2A and 2B depict tables and graphical representation of results from a microbial treatment of organic chemical agents as an example of the method of the disclosure for an embodiment used for soil remediation.

### DETAILED DESCRIPTION

The following description and drawings are illustrative of the disclosure and are not to be construed as limiting the disclosure. Numerous specific details are described to provide a thorough understanding of various embodiments of the present disclosure. However, in certain instances, well-known or conventional details are not described in order to provide a concise discussion of embodiments of the present disclosure.

Various apparatuses or processes will be described below to provide examples of embodiments of the treatment method and system disclosed herein. No embodiment described below limits any claimed embodiment and any claimed embodiments may cover processes or apparatuses that differ from those described below. The claimed embodiments are not limited to apparatuses or processes having all of the features of any one apparatus or process described below or to features common to multiple or all of the apparatuses or processes described below.

Furthermore, numerous specific details are set forth in order to provide a thorough understanding of the embodiments described herein. However, it will be understood by those of ordinary skill in the art that the embodiments described herein may be practiced without these specific details. In other instances, well-known methods, procedures and components have not been described in detail so as not to obscure the embodiments described herein.

Unless defined otherwise, all technical and scientific terms used herein are intended to have the same meaning as commonly understood to one of ordinary skill in the art.

A method and composition is provided in the present disclosure for cleaning animal drinking water or maintaining clean animal drinking water, by treatment with sporulated microbes. The sporulated microbes provide a stable format that is temperature and pressure resistant. Upon exposure to the drinking water, the sporulated bacteria are released and activated, and metabolize organic matter in the drinking water, thus cleaning the drinking water.

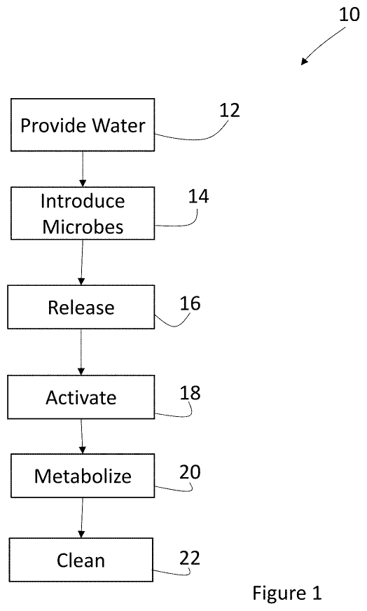

As shown in the overview of FIG. 1, the method 10 comprises providing animal drinking water 12 in a container. Sporulated microbes are introduced 14 into the animal drinking water. In one embodiment, the microbes are released 16 and activated 18. The microbes metabolize 20 organic matter in the water thus cleaning 22 the drinking water.

Sporulated microbes form in response to inhospitable conditions, such as a severe lack of nutrients, that would be lethal for the normal (vegetative) form of the microbe. In the case of bacteria, the bacteria respond by producing and activating a protein that promotes the transcription of genes required for the conversion of the actively growing bacterium to a spore. During sporulation, the bacterial genome is duplicated and the second copy of the genome is enveloped together with some cytoplasm within an in-growth of the bacterial cell well. The resultant daughter cell is stabilized by formation of another membrane layer, a peptidoglycan material between the two membrane layers, and an outer coat of proteins, thereby forming an almost impregnable sphere. The mother cell dies and degrades, freeing the spore, which remains dormant until the environment becomes hospitable and the spore transforms back into a vegetative cell. Sporulated bacteria are stable throughout a range of conditions, which in the present disclosure provides an improved and advantageous format for the present application.

Bacteria are induced to be in the sporulated state by culture at high density (for example at 1.0×10¹¹ CFU/Gallon) in a media lacking nutrients, such as Tryptone, Soy and Proteins. The sporulated bacteria can be activated to come out of the dormant state in the presence of appropriate nutrients for the sporulated bacteria and/or in the presence of a higher pH.

To further enhance the beneficial effects of the sporulated microbes, other agents can be added with the microbes. These include an anti-pathogen agent to mitigate pathogens, such as protozoan parasites, helminth parasites, zoonotic pathogens and coliform bacteria. Additional agents include an animal digestive system enhancement agent (for example bacteria for digesting cellulose and lignan), an animal microbiome stability agent, an animal immune system support agent and a water contaminant mitigation agent. These additional agents are provided to the drinking water together with the sporulated microbes.

Water contamination mitigation agents may include nitrifying bacteria for oxidizing ammonia to nitrites and nitrites to nitrates. Water treatment using microbes can incorporate cleaning bacteria and nitrifying bacteria. Cleaning bacteria digest decomposing organic material, which produces ammonia, and assists nitrifying bacteria by preventing loss of oxygen caused by the decomposing material. Nitrifying bacteria include ammonia oxidizing bacteria that convert ammonia into nitrites, and nitrite oxidizing bacteria that convert nitrite into nitrate.

Water contamination mitigation agents may also include chelating agents for chelating metals and metalloids, such as arsenic and aluminum. Other water contamination mitigation agents are bacteria for metabolizing pesticides or herbicides, such as glyphosate, Dicamba, 2,4-dichlorophenoxyacetic acid, Atrazine, Fomesafen, Imidacloprid, Clothianidin or Thiamethoxam.

The sporulated microbes may be provided in a format such as a pellet, a tablet, a granule, a bolus, a spike, a mash, a crumble, a meal, a liquid, or a powder. In the case of a liquid, the sporulated microbes are at least 1×10¹¹ CFU/Gallon and a pH lower than 8.5, which maintains the sporulated bacteria in a dormant state.

In an embodiment of the present disclosure, the sporulated microbes are at least two of Bacillus amyloliquefaciens, Bacillus subtilis, Rhodopseudomonas palustris, and Saccharomyces cerevisiae.

To accomplish a preferred release rate or dosing rate, the sporulated microbes are adsorbed or packed with a carrier or provided in a format such as a tablet. The packing or carrier affects the rate of release of the microbes by providing a level of water accessibility or effervescence. With greater water accessibility or effervescence of the carrier, the microbes are released faster, and with lower water accessibility or effervescence, the release is slower and can be extended to at least 24 hours. The release rate can determine the CFU count in the treated drinking water, so different carriers can be utilized for different CFU concentration in the drinking water and different dosing characteristics. In addition, faster release tends to enable a greater concentration of the sporulated microbes and other additional agents towards the top of the water column of the drinking water, whereas slower release tends to enable a greater concentration of the sporulated microbes and additional agents towards the bottom of the water column of the drinking water. Slower release of the sporulated microbes also has an advantage of extending the water treatment time and requiring fewer additions of the treatment. Examples of carriers are salts and inert minerals such as zeolite and calcium byproducts.

In a further embodiment, a ratio of aerobic to anaerobic microbes is adjusted to target the condition of the water to be treated and attain a minimum CFU/Gallon of microbes in the drinking water. The ratio can be adjusted to take into account the water depth and level in the water column that is being targeted for treatment. Aerobic microbes will inhabit areas closer to the water surface, while anaerobic microbes will inhabit areas at greater water depth. Thus by providing a higher aerobic to anaerobic microbe ratio, a larger proportion of the microbes will be concentrated higher in the water column. Conversely, a lower aerobic to anaerobic microbes ratio will result in a larger proportion of the microbes lower in the water column. The anaerobic microbes are more effective in reducing organic matter. The aerobic microbes are more effective in treating adverse chemical conditions of the drinking water and are advantageous for animals to drink, for example to reduce coliforms. Thus the ratio of aerobic to anaerobic microbes can be customized to the water treatment required.

A composition for treating animal drinking water includes sporulated microbes as described above. The composition may further include an anti-pathogen agent, an animal digestive system enhancement agent, an animal microbiome stability agent, an animal immune system support agent, and/or a water contamination mitigation agent. Examples of a water contamination mitigation agent are a nitrifying bacteria, a chelating agent for a metal or a metalloid, and a bacteria for metabolizing a pesticide.

The composition for treating animal drinking water may be in the form of a pellet, tablet, granule, bolus, spike, mash, crumble, meal, liquid or powder. In liquid form, the liquid may preferentially be a solution with a concentration of the sporulated microbes of at least 1×10¹¹ CFU/gallon and a pH lower than 8.5.

Sporulated microbes that may be included in the composition are at least two of Bacillus amyloliquefaciens, Bacillus subtilis, Rhodopseudomonas palustris, and Saccharomyces cerevisiae. The composition may also include a carrier for the sporulated microbes. The characteristics of the carrier may be used to regulate the release rate of the microbes into the drinking water. The microbe composition may also be varied in the ratio of aerobic to anaerobic microbes, which would vary the distribution of the microbes once released into the drinking water.

### Alternative Embodiment for Soil Remediation

An object of an alternative embodiment discussed herein is to provide a system and method for breakdown of agricultural products using beneficial microbes.

Thus by one broad aspect of the present invention, a method for breaking down an organic chemical agent is provided, comprising providing a plurality of sporulated microbes to the chemical agent.

By a further broad aspect of the present invention, a composition for breaking down an organic chemical agent is provided, comprising a plurality of sporulated microbes with a density of at least 1×10¹¹ CFU/Gallon.

“Organic chemical agent” as used herein means any carbon chain containing chemical, including but not limited to herbicides and petroleum products.

“Microbes” as used herein include bacteria, fungi, algae, protozoa and viruses.

Organic chemical agents can be broken down by microbes, including a mixture of beneficial bacteria. In order to stably formulate beneficial bacteria, the bacteria are sporulated to provide a stable format that is resistant to damage and is not active until distribution, for example distribution on plants or soil.

Sporulated bacteria form in response to inhospitable conditions, such as a severe lack of nutrients, that would be lethal for the normal (vegetative) form of the bacteria. The bacteria respond by producing and activating a protein that promotes the transcription of genes required for the conversion of the actively growing bacterium to a spore. During sporulation, the bacterial genome is duplicated and the second copy of the genome is enveloped together with some cytoplasm within an in-growth of the bacterial cell wall. The resultant daughter cell is stabilized by formation of another membrane layer, a peptidoglycan material between the two membrane layers, and an outer coat of proteins, thereby forming an almost impregnable sphere. The mother cell dies and degrades, freeing the spore, which remains dormant until the environment becomes hospitable and the spore transforms back into a vegetative cell. Sporulated bacteria are stable throughout a range of conditions, which in the present disclosure provides an improved and advantageous format for the present application.

Bacteria are induced to be in the sporulated state by culture at high density (for example at 1.0×10¹¹ CFU/Gallon) in a media lacking nutrients, such as Tryptone, Soy and Proteins. The sporulated bacteria can be activated to come out of the dormant state in the presence of appropriate nutrients for the sporulated bacteria and/or in the presence of a higher pH.

Organic chemical agents may be an agricultural agent or a petroleum agent. Agricultural agents are, for example, herbicides, such as: glyphosate; Dicamba; 2,4-Dichlorophenoxyacetic acid (also called 2,4-D); Atrazine; and Fomesafen herbicide; or insecticides such as: Imidacloprid; Clothianidin; and Thiamethoxam.

The treatment of a chemical agent may be done by adding the mixture of sporulated microbes to the organic chemical agent prior to distribution on plants or soil. The sporulated bacteria do not degrade the organic chemical agent prior to distribution because they remain in the dormant state. The sporulated bacteria remain dormant in the presence of the chemical agent until approximately 24 hours after the distribution of the mixture onto, for example, plants, such as agricultural crops or onto soil. The lag time of approximately 24 hours for the sporulated bacteria to become active allows the chemical agent, which is typically active within one hour of application, to achieve its function as a herbicide or insecticide, before being degraded by the beneficial bacteria regenerated from the sporulated bacteria.

Alternatively, the treatment of a chemical agent can be done by distributing the microbe mixture onto plants after the plants have been treated with the chemical agent.

A further method is to treat the chemical agent by distributing the microbe mixture on soil that has been treated with the chemical agent, or on soil that is contaminated with the chemical agent.

After distribution onto, for example, plants or soil, the activated bacteria degrade the chemical agent by breaking down the carbon chain of the chemical agent or other covalent bonds that are metabolic targets of the active bacteria. Breakdown of the chemical bonds of the chemical agent may be carried out by enzymes secreted by the active bacteria. A mixture of at least two bacterial species is used to provide a wider spectrum of active proteins and enzymes to metabolize the organic chemical agent. The bacterial mixture also works synergistically such that one bacterial species may degrade metabolic products created by another bacterial species. In addition, enzymes secreted by one bacterial species may target degradation products of the organic chemical agent. Thus the combination of bacterial species provides greater activity than a simple additive advantage of individual species.

The sporulated microbes can be in a pellet or a powder form. The sporulated microbes can also be in a liquid format, at a pH lower than 8.5 or ideally at a pH between 4.5 and 5.5. In this format, the sporulated microbes do not degrade the organic chemical. Thus they can be mixed with the organic chemical agent prior to activation of the sporulated microbes, without degradation of the organic chemical. This provides a useful format to package an organic chemical, such as a herbicide or insecticide, together with the dormant sporulated microbes, prior to application, for example to agricultural crops.

The sporulated microbes may be a mixture of at least two of the following bacterial species: Bacillus licheniformis; Bacillus coagulans; Bacillus subtilis; Bacillus pumilus; Bacillus megaterium; and Bacillus amyloliquefaciens.

In a further embodiment, cellulose- and lignan-composting bacteria may be included in the microbe mixture, to break down dying material after a crop has been harvested and/or to break down weed waste.

The mixture of sporulated microbes may also include any of a nutrient, an extract or a sugar to improve microbial performance. For example, the sporulated microbe mixture may include seaweed extract.

An example embodiment of the present disclosure is provided below, including customer directions for a biological soil amendment for bio-remediation of pesticides and petroleum spills and residues.

In the example embodiment the product consists of a concentrated microbial powder which is a highly effective and concentrated biological amendment containing a broad spectrum of microbials specially selected for pesticide and petroleum remediation. The concentrated package of bacteria can be sprayed with water or mixed with all fertilizer types, bio-stimulants, or adjuvants to effectively and efficiently remove pesticide and petroleum residues and spills from soils.

Directions for use of the powder for broadcast or band boom applications: mix the concentrated powder in water soluble bag in spray tank that is half full. Add remaining mixture to fill tank. Mix thoroughly in tank before spraying. Spray volume per acre should be no less than 15 gallons per acre of finished spray. The product is compatible with all insecticides, herbicides, fungicides, adjuvants and fertilizers. Do not mix into a product that has a pH higher than 8.5. Alkaline products should be mixed with water to dilute the pH below 8.5 before adding the powder.

Directions for use of the powder for irrigation applications: mix the powder at well site in container to allow water soluble bags to dissolve and be mixed thoroughly before pumping through irrigation system. Can be applied with drip or foliar irrigation equipment. Do not apply with flood in-furrow irrigation. Can be applied in conjunction with all fertilizers or micronutrients formulations.

Application rates for the powder: half pound water soluble bag with concentrated powder will treat 5 acres at a final concentration of 750 billion CFU/Acre

A two-pound packet with four half pound bags treats 20 acres with final concentration of 750 billion CFU/Acre

For this embodiment, Four to six applications per season recommended for optimum results. Applying in conjunction with humic acids or other microbial food sources can be beneficial and recommended in low organic matter soils.

In one embodiment, the powder includes 75.50% inert ingredients and 24.5% of non-plant food ingredients, including the following soil health microbes:

Bacillus licheniformis—5.8×10⁸ CFU/Gm

Bacillus coagulans—5.8×10⁸ CFU/Gm

Bacillus subtilis—5.8×10⁸ CFU/Gm

Bacillus pumilus—5.8×10⁸ CFU/Gm

Bacillus megaterium—5.8×10⁸ CFU/Gm

Bacillus amyloliquefaciens—5.8×10⁸ CFU/Gm

In one embodiment, the powder has a five-year shelf life if kept dry and does not require refrigeration. The end user is to follow SDS instructions for safety precautions, cleaning and PPE (Personal Protective Equipment). If no such instructions for washables, use detergent and hot water.

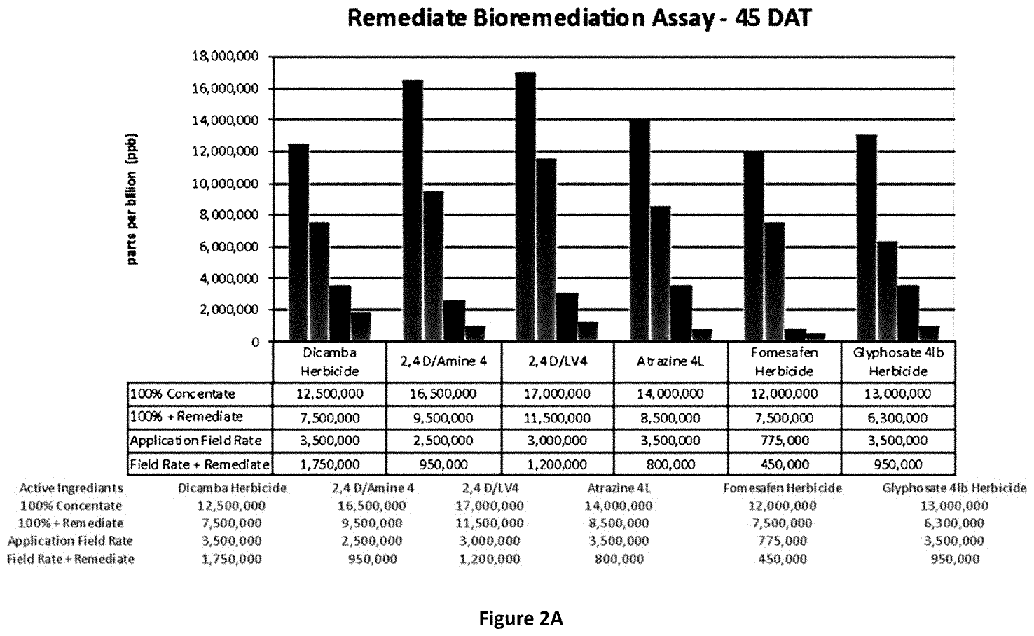

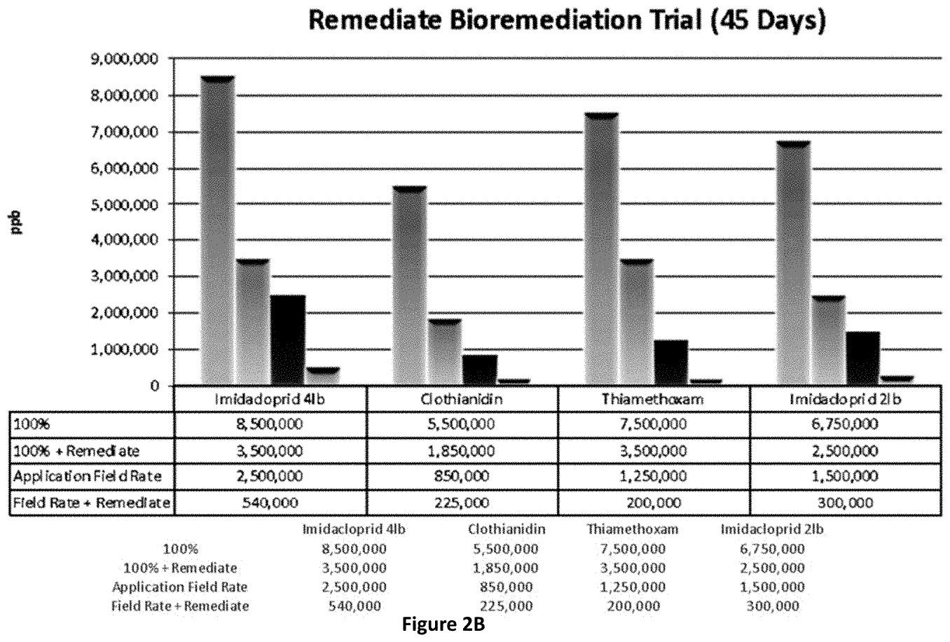

An example of the treatment of a variety of herbicides and insecticides is presented in FIGS. 2A and 2B. In FIGS. 2A and 2B, each set of four bar codes represents the values for 100% Concentrate, 100%+Remediate, Application Field Rate, and finally Field Rate+Remediate.

Chemical treatments, including the herbicides: glyphosate; Dicamba; 2,4-Dichlorophenoxyacetic acid (also called 2,4-D); Atrazine; and Fomesafen herbicide; and the insecticides: Imidacloprid; Clothianidin; and Thiamethoxam, were added alone or in combination with the sporulated microbe mixture. Samples were taken 45 days following drenching of herbicides and analyzed for residual herbicide using a Remediation Assay. Results are shown for no treatment, treatment, soil treatment with chemical alone, and soil treatment with chemical and microbes. With one application, the microbes are able to reduce the level of organic chemical by 50% in 45 days.

A composition for treatment of an organic chemical agent is made up of sporulated microbes with a density of at least 1×10¹¹ CFU/Gallon. The microbes are dormant in the composition, but able to be activated when the pH raises above 5.5 and/or when the bacterial spores are exposed to nutrients. The microbes in the composition may contain at least two of: Bacillus licheniformis; Bacillus coagulans; Bacillus subtilis; Bacillus pumilus; Bacillus megaterium; and Bacillus amyloliquefaciens.

In a further embodiment, the composition may include cellulose- and lignan-composting bacteria in the microbe mixture, to break down dying material after a crop has been harvested and/or to break down weed waste.

While the applicant's teachings described herein are in conjunction with various embodiments for illustrative purposes, it is not intended that the applicant's teachings be limited to such embodiments. On the contrary, the applicant's teachings described and illustrated herein encompass various alternatives, modifications, and equivalents, without departing from the embodiments, the general scope of which is defined in the appended claims. Except to the extent necessary or inherent in the processes themselves, no particular order to steps or stages of methods or processes described in this disclosure is intended or implied. In many cases the order of process steps may be varied without changing the purpose, effect, or import of the methods described.

What is claimed is:

1. A method for treating drinking water for animals, comprising providing a plurality of sporulated microbes to metabolize an organic matter in the drinking water.

2. The method as in claim 1, further comprising adding one or more of:
an anti-pathogen agent to mitigate pathogens;
an animal digestive system enhancement agent;
an animal microbiome stability agent;
an animal immune system support agent; and
a water contaminant mitigation agent;
wherein the one or more agents are provided to the drinking water with the plurality of sporulated microbes.

3. The method as in claim 2, wherein the water contaminant mitigation agent comprises one or more of:
a nitrifying bacteria;
a chelating agent for a metal or a metalloid; and
a bacteria for metabolizing a pesticide.

4. The method as in claim 1, wherein the plurality of sporulated microbes comprises:
a pellet;
a tablet;
a granule;
a bolus;
a spike;
a mash;
a crumble;
a meal;
a liquid; or
a powder.

5. The method as in claim 4, wherein the liquid comprises a solution with a concentration of the plurality of sporulated microbes at least 1×10¹¹ CFU/Gallon and a pH lower than 8.5.

6. The method as in claim 1, wherein the sporulated microbes comprise at least two of:
Bacillus amyloliquefaciens;
Bacillus subtilis;
Rhodopseudomonas palustris; and
Saccharomyces cerevisiae.

7. The method as in claim 1, further comprising adsorbing the plurality of microbes to a carrier to regulate a release rate of the plurality of microbes into the drinking water.

8. The method as in claim 1, further comprising providing a ratio of aerobic to anaerobic microbes to achieve a target distribution of the microbes in the drinking water.

9. A composition for treating animal drinking water, comprising a plurality of sporulated microbes.

10. The composition as in claim 9, further comprising at least one of:
an anti-pathogen agent;
an animal digestive system enhancement agent;
an animal microbiome stability agent;
an animal immune system support agent; and
a water contamination mitigation agent.

11. The composition as in claim 10, wherein the water contamination mitigation agent comprises one or more of
a nitrifying bacteria;
a chelating agent for a metal or a metalloid; and
a bacteria for metabolizing a pesticide.

12. The composition as in claim 9, wherein the plurality of sporulated microbes comprise:
a pellet;
a tablet;
a granule;
a bolus;
a spike;
a mash;
a crumble;
a meal;
a liquid; or
a powder.

13. The composition as in claim 12, wherein the liquid comprises a solution with a concentration of the plurality of sporulated microbes at least 1×10¹¹ CFU/Gallon and a pH lower than 8.5.

14. The composition as in claim 9, wherein the sporulated microbes comprise at least two of:
Bacillus amyloliquefaciens;
Bacillus subtilis;
Rhodopseudomonas palustris; and
Saccharomyces cerevisiae.

15. The composition as in claim 9, further comprising a carrier for adsorbing the plurality of microbes, for regulating a release rate of the plurality of microbes into the drinking water.

16. The composition as in claim 9, further comprising a ratio of aerobic to anaerobic microbes to achieve a target distribution of the microbes in the drinking water.
